(19) United States
(12) Patent Application Publication
(10) Pub. No.: US 2013/0164342 A1
(43) Pub. Date: Jun. 27, 2013

(54) FORMULATIONS FOR OSTEOPOROSIS

(71) Applicant: Mahmut Bilgic, Istanbul (TR)

(72) Inventor: Mahmut Bilgic, Istanbul (TR)

(21) Appl. No.: 13/692,163

(22) Filed: Dec. 3, 2012

Related U.S. Application Data
(63) Continuation-in-part of application No. PCT/TR2011/000150, filed on Jun. 2, 2011.

(30) Foreign Application Priority Data
Jun. 3, 2010 (TR) TR 2010/04464
Dec. 9, 2011 (TR) TR 2011/12197

(51) Int. Cl. A61K 9/46

(52) U.S. Cl. CPC A61K 9/0007
USPC 424/400; 424/44; 424/682; 424/687

(57) ABSTRACT

The present invention relates to effervescent formulations comprising genistein with an average particle size (d₅₀) in the range of 1 to 300 μm as active agent. The present invention also relates to pharmaceutical formulations with high bioavailability comprising calcium, genistein and vitamin D in order to be used in prophylaxis and treatment of osteoporosis and related diseases.

### CROSS-REFERENCE TO RELATED APPLICATIONS

This application is a continuation-in-part of PCT/TR2011/000150, filed Jun. 2, 2011 and claims foreign priority to application Serial Number TR2011/12197, filed Dec. 9, 2011, which are incorporated herein by reference in their entireties. This application is entitled to and claims priority benefits to application Serial Number TR2010/04464, filed Jun. 3, 2010.

### BACKGROUND OF THE INVENTION

Bone loss, or in other terms “osteoporosis”, is defined as a disease characterized by liability to bone fracture and increased risk of bone fracture as a consequence of low bone mass and deterioration of bone micro architecture. The disease does not generally result in death; however, it is a major health problem affecting life quality.

Although osteoporosis is mostly seen in spine, hip and wrist; all the bones in the body are affected by the disease. 80% of the patients are female though the disease is encountered in both genders. Postmenopausal women lose 0.7-2% of total bone mass annually while this ratio is 0.5-0.7% for men. In parallel with this, total bone mass loss in men and women between 45-70 years of age is 15-30%. Some studies conducted nowadays indicate that insufficient calcium intake in childhood may increase the risk of osteoporosis later in life.

It is known that calcium taken by diet plays a significant role in prophylaxis of osteoporosis. However, calcium absorption is reduced in postmenopausal term due to physical features of calcium and other factors. One of the most important problems encountered in calcium intake is that calcium salts in all calcium sources do not dissolve at an equal rate or present desired bioavailability. Calcium salts have low water solubility. They are dissolved relatively better in gastric acid; however, carbon dioxide produced causes gas and pain in the stomach. Furthermore, gastric acid is reduced due to gastric acid inhibitors used in later ages and therefore calcium leaves the stomach without being dissolved.

Another factor for reduced calcium absorption in postmenopausal women is vitamin D deficiency. Vitamin D is a fat-soluble hormone precursor and the studies conducted have indicated that efficient amounts of vitamin D increases calcium absorption. Vitamin D deficiency is also seen in individuals who do not receive sufficient sunlight.

Various methods and formulations have been developed so as to prevent osteoporosis, to decrease the risk of osteoporosis occurrence and to treat osteoporosis; yet, none of these have attained to desired efficiency. It has been observed that use of calcium together with sex hormones, particularly with oestrogen is quite effective in preventing loss of bone mass but it has not been a frequently preferred method since this type of hormones facilitate development of carcinoma (breast, endometrial, ovary cancer). Although risk of carcinoma occurrence is less in calcium and oestrogen/progesterone treatment, there are still serious problems in this method, too. Researchers indicated that calcitonin, which is a calcium regulating hormone, can be used in osteoporosis treatment, particularly in postmenopausal osteoporosis treatment in the cases where oestrogen treatment is not suitable. Though yielded successful results, use of this treatment method has remained limited as the patients started to resist this treatment and it is high cost. Considering these studies conducted, it is clear that various hormones contribute to calcium intake. However, calcium and hormone combination has not been preferred due to the reasons such as side effects, resistance and cost.

Nevertheless, with the use of genistein that is a phytoestrogen instead of oestrogen in recent years, bone mass could be increased without occurrence of the side effects observed in other hormone therapies. Moreover, it has been proved that genistein alleviates the symptoms observed in postmenopausal term such as hot flash and night sweat.

The studies conducted have indicated that use of vitamin D and genistein together is more effective compared with use of these agents separately or as dual combination.

With confirmation of this efficiency, the inventors have developed formulations including calcium, vitamin D, and genistein. Tablet formulation including calcium, vitamin D, and genistein that exists on the market now has been developed and put on the market in line with this purpose.

However, it frequently poses problems to administer solid dosage forms such as pills, tablets, capsules by the oral route in elderly patients. Due to the problems appearing particularly in pharyngeal and esophageal movements, the individuals may have a feel of obstruction or asphyxiation, and as a result, intake of the medicament may turn disgustful and painful for the patient. Another disadvantage of tablet formulations including calcium is gas and pain in the stomach resulting from carbon dioxide produced while calcium is being dissolved in gastric acid. Furthermore, calcium leaves the stomach without being dissolved due to reduction of gastric acid in patients using gastric acid inhibitors and thus, the patient cannot benefit sufficient bioavailability.

When the prior art is taken into consideration, there is need for development of user-friendly formulations which include calcium, vitamin D, and genistein; do not present the side effects such as gas and pain in the stomach that existing formulations pose in use; and have high bioavailability in order to be used in the prophylaxis and treatment of osteoporosis and related diseases.

Plant-derived estrogens or phenolic compounds with an effect similar to estrogen are called phytoestrogens. Phytoestrogens, which have various degrees of estrogenic activity and resemble natural estrogens in terms of both structure and shape, can induce a direct effect binding to receptors after competing with the natural estrogens present in the body. Besides, phytoestrogens may change the activity of some enzymes playing role in the estrogen metabolism.

Phytoestrogens are divided into different sub-classes according to various sources. These sub-classes can be four in number as isoflavones, lignans, coumestans, and stilbenes. Each class includes different compounds and the sources of these compounds in the human food vary.

Genistein is a compound belonging to the isoflavone class of flavonoids present in plants. Besides, as isoflavones are plant-derived compounds with a biological effect similar to estrogens, they are also called phytoestrogens. Isoflavones are mainly present in soybeans and soy products. 100 g soybean includes 111 mg genistein.

In the recent years, there are many studies conducted for the potential protective effects of isoflavones against cardiovascular diseases, menopause syndromes; bone diseases like osteoporosis, osteomalacia, and fibrous osteodystrophy; and hormonal cancers (breast and prostate cancer).

In the prior art, there are many applications about how to obtain the compounds. For example, the application numbered U.S. Pat. No. 512,192 discloses the production of genistein from a soy product substrate by fermentation.

The application numbered WO99/38509 discloses a genistein formulation produced by micro-emulsion method.

However, the prior art does not mention the use of this plant extract as a pharmaceutically effective dosage form.

### SUMMARY OF THE INVENTION

The invention features formulations including calcium, vitamin D, and genistein, e.g., in effervescent form.

The formulation can be, e.g., formulated in powder, granule, pellet, micro tablet or tablet form.

The calcium included in the formulation can be, e.g., in salt form, e.g., in carbonate, chloride, phosphate, citrate, lactate, glubionate, gluceptate, or gluconate salt form. In certain aspects, the formulation includes 5-60% calcium or calcium salt by weight and/or the amount of calcium in the formulation can be in the range of 200-2000 mg. The particle size of calcium or calcium salt in the formulation can be, e.g., smaller than 100 μm, e.g smaller than 60 μm.

The vitamin D in the formulation can be, e.g., vitamin D₂ (ergocalciferol), vitamin D₃ (cholecalciferol), or a combination thereof. The formulation can include, e.g., 0.01-15% vitamin D by weight and/or the amount of vitamin D in the formulation can be, e.g., in the range of 200-1000 IU.

The formulation can include, e.g., 0.1-25% genistein by weight. For example, the amount of genistein in the formulation can be in the range of 10-70 mg, preferably in the range of 20-55 mg.

The formulation of the present invention can also include, e.g., pharmaceutically acceptable excipients in addition to calcium, vitamin D, and genistein. In some embodiments, one or more excipients can include, e.g., an effervescent couple, diluent (e.g., lactose, maltose, dextrin, maltodextrin, mannitol, sorbitol, and starch, or a combination thereof), binder, glidant, lubricant, disintegrant, flavoring agent, sweetener, coloring agent, surfactant, anti-foam agent, or stabilizing agent.

The effervescent couple can be, e.g., composed of an effervescent acid (e.g., acetic acid, citric acid, lactic acid, malic acid, phosphoric acid, propionic acid, and tartaric acid, or combinations thereof) and an effervescent base (e.g., potassium carbonate, potassium bicarbonate, potassium citrate, potassium hydroxide, sodium carbonate, sodium hydrogen carbonate, and sodium hydrogen sulfate, or combinations thereof). The formulation can include, e.g., less than 15% diluents by weight.

The binder can, e.g., be starches such as potato starch, corn starch, wheat starch; sugars such as, e.g., sucrose, glucose, dextrose, lactose, maltodextrin; natural and synthetic gums; gelatin; cellulose derivatives such as, e.g., microcrystalline cellulose, HPC, HEC, HPMC, carboxymethyl cellulose, methyl cellulose, ethyl cellulose; polyvinylpyrrolidone (povidone), polyethylene glycol (PEG); waxes; calcium carbonate; calcium phosphate; alcohols such as, e.g., sorbitol, xylitol, mannitol, water, or a combination thereof. In some embodiments, the binder is lactose, povidone, polyethylene glycol, or a combination thereof.

If a glidant is present, the glidant can be, e.g., sodium lauryl sulfate, sodium benzoate, sodium chloride, sodium acetate, sodium fumarate, carbowax 4000, L-leucine(17), PEG, or a combination thereof. If a lubricant is present, the lubricant can be, e.g., talc, magnesium stearate, stearic acid, sodium stearyl fumarate, polyoxyethylene glycol, leucine, alanine, glycine, sodium benzoate, sodium acetate, or fumaric acid, or a combination thereof. If a disintegrant is present, the deisintegrant can be, e.g., potato starch, corn starch, wheat starch, pregelatinized starch, sodium starch glycolate; cellulose derivatives such as croscarmellose sodium or microcrystalline cellulose; polyvinylpyrrolidone; crospovidone; alginic acid and its salts; clays such as, e.g., xanthan gum or Veegum; ion exchange resins, or a combination thereof.

In certain embodiments, the flavoring agent can be, e.g., natural aroma oils (peppermint oil, wintergreen oil, clove bud oil, parsley oil, eucalyptus oil, lemon oil, orange oil), menthol, menthane, anethole, methyl salicylate, eucalyptol, cinnamon, 1-methyl acetate, sage, eugenol, oxanone, alpha irisone, marjoram, lemon, orange, blackberry, propenyl guaetol acetyl, cinnamon, vanilla, thymol, linalol, cinnamaldehyde glycerol acetal, N-substituted p-menthane-3-carboxamide, or 3,1-methoxy propane 1,2-diol, or a combination thereof.

If a sweetener is present, it can be, e.g., sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D-tryptophane, monoammonium glycyrrhizinate, neohesperidin dihydrochalcone, thaumatin, neotame, alitame, stevioside, cyclamates, or a combination thereof. If a coloring agent is present, it can be, e.g., a carotenoid, chlorophyl, or a combination thereof. If a surfactant is present, it can be, e.g., sodium lauryl sulfate, magnesium lauryl sulfate, or a combination thereof. If an anti-foam agent is present, it can be, e.g., simethicone emulsion, dimethyl siloxane, silicon oil, or a combination thereof. If a stabilizing agent is present, it can be, e.g., an antioxidant, chelating agent, an alkalinizing agent, or a photoprotective agent.

The above formulations can include, e.g., a diluent in the range of 0.01-15% by weight, an effervescent couple in the range of 20-90% by weight, and other excipients in the range of 0.1-15% by weight.

In all aspects of the invention, the formulation can be used to treat diseases such as, e.g., osteoporosis; bone fracture including vertebral column and hip bones in postmenopausal women; bone fracture in men; idiopathic osteoporosis; osteoporosis resulting from various diseases; steroid and glucocorticoid-induced osteoporosis; osteopenia; osteomalacia; osteogenesis imperfecta; osteochondrodisplasia; sudeck atrophy; rheumatoid arthritis; Paget's disease; metastasis of malignant tumors to bones; hypercalcemia; or hyperthyroidism.

In another aspect, the invention also features an effervescent formulation, characterized in that the formulation can include genistein, e.g., with an average particle size (d₅₀) in the range of 1 to 300 μm as active agent (e.g., 1 to 250 μm), at least one pharmaceutically acceptable effervescent acid (e.g., acetic acid, citric acid, lactic acid, malic acid, phosphoric acid, propionic acid, tartaric acid or hydrates, anhydrates, or combinations thereof), at least one pharmaceutically acceptable effervescent base (e.g., potassium carbonate, potassium bicarbonate, potassium citrate, potassium hydroxide, sodium carbonate, sodium hydrogen carbonate, sodium hydrogen citrate, or combinations thereof), and at least another excipient (e.g., diluents, binders, sweeteners, lubricants, solvents, flavoring agents, pH regulating agents, coloring agents, or combinations thereof).

The one or more effervescent bases can be, e.g., in the range of 1 to 70% or 1 to 50% by weight (e.g., 1% by weight). The one or more effervescent acids can be, e.g., present at least at 20% by weight (e.g., 20 to 80% by weight or 20 to 75% by weight). The genistein can be, e.g., at least 0.1% by weight (e.g., 0.1 to 5% by weight or 0.1 to 2% by weight).

The above effervescent formulation can include, e.g., a second active agent. In certain embodiments, the second active agent is, e.g., a mineral, such as calcium, potassium, magnesium, iron, sodium, zinc; or their salts, such as, e.g., carbonate or sulphate; vitamins such as, e.g., vitamin A, vitamins B such as B1, B12, B6 and/or folic acid, vitamin C, vitamin D, and vitamin E, or combinations thereof.

In some aspects, the invention features a production method for the production of the above effervescent formulation, e.g., a wet granulation method.

In all aspects of the invention, the formulations can be used, e.g., in the treatment and/or prevention of bone diseases particularly such as, e.g., osteoporosis, osteomalacia, and fibrous osteodystrophy, e.g., those observered in the postmenopausal period.

### DESCRIPTION OF THE INVENTION

The inventors have surprisingly found that effervescent formulations including calcium, vitamin D and genistein are more user-friendly, have fewer side effects and present higher bioavailability compared with tablet formulations; therefore, they display higher efficacy in the prophylaxis and treatment of osteoporosis and related diseases in comparison with existing formulations.

In this respect, the present invention relates to effervescent formulations including calcium, vitamin D and genistein.

Effervescent formulation of the present invention can be in powder, granule, pellet, micro tablet or tablet form though it is preferably in tablet form.

Calcium used in the formulation of the present invention is preferably in salt form.

Calcium used in the formulation of the present invention can be selected from calcium, carbonate, chloride, phosphate, citrate, lactate, glubionate, gluceptate, gluconate salts though it is preferably carbonate salt.

Formulation of the present invention can include 5-60% calcium or calcium salt by weight.

Formulation of the present invention can include 200-2000 mg calcium or calcium salt in an equal amount to this.

Considering that calcium salts have low water solubility in general, the inventors have encountered solubility problems in development of effervescent dosage forms including calcium or calcium salt. The inventors could attain to efficient solubility when they used calcium or calcium salt with a particle size smaller than 100 μm, preferably smaller than 60 μm. In this aspect, the present invention relates to effervescent formulations including calcium or calcium salt with a particle size smaller than 100 μm, preferably smaller than 60 μm, vitamin D and genistein.

Vitamin D included in the formulation of the present invention can be vitamin D₂ (ergocalciferol) or vitamin D₃ (cholecalciferol) though it is preferably vitamin D₃ (cholecalciferol).

Formulation of the present invention can include 0,01-15% vitamin D by weight. Formulation of the present invention can include 200-1000 IU vitamin D. Formulation of the present invention can include 0,1-25% genistein by weight. Formulation of the present invention can include 10-70 mg, preferably 20-55 mg genistein.

The formulation of the present invention can include an effervescent couple to provide water solubility characteristic. The term “effervescent couple” refers to use of an acidic agent and a basic agent together.

The pharmaceutically acceptable acidic agent of the present invention can be selected from a group including acetic acid, citric acid, lactic acid, malic acid, phosphoric acid, propionic acid, tartaric acid or combinations thereof.

The pharmaceutically acceptable acidic agent used in the formulation of the present invention is preferably citric acid, malic acid or a combination thereof.

The pharmaceutically acceptable basic agent of the present invention can be selected from a group including potassium carbonate, potassium bicarbonate, potassium citrate, potassium hydroxide, sodium carbonate, sodium hydrogen carbonate, sodium hydrogen citrate or combinations thereof.

The pharmaceutically acceptable basic agent used in the formulation of the present invention is preferably sodium carbonate, sodium hydrogen carbonate or a combination thereof.

The effervescent formulation of the present invention can optionally include one or more of the excipients including diluent, binder, glidant, lubricant, disintegrant, flavoring agent, sweetener, coloring agent, surfactant, anti-foam agent, stabilizing agents.

The diluents can be selected from a group including lactose, maltose, dextrin, maltodextrin, mannitol, sorbitol, starch or a combination thereof.

The inventors have found that solubility rate is increased when they use less than 15% diluent by weight in the formulation.

The binder can be selected from a group including starches such as potato starch, corn starch, wheat starch; sugars such as sucrose, glucose, dextrose, lactose, maltodextrin; natural and synthetic gums; gelatin; cellulose derivatives such as microcrystalline cellulose, HPC, HEC, HPMC, carboxymethyl cellulose, methyl cellulose, ethyl cellulose; polyvinylpyrrolidone (povidone), polyethylene glycol (PEG); waxes; calcium carbonate; calcium phosphate; alcohols such as sorbitol, xylitol, mannitol, and water or a combination thereof though it is preferably lactose, povidone, polyethylene glycol or a combination thereof.

The glidant can be selected from a group including sodium lauryl sulfate, sodium benzoate, sodium chloride, sodium acetate, sodium fumarate, carbowax 4000, L-leucine(17), PEG or a combination thereof.

The lubricant can be selected from a group including talc, magnesium stearate, stearic acid, sodium stearil fumarate, polyoxyethylene glycol, leucine, alanine, glycine, sodium benzoate, sodium acetate, fumaric acid or a combination thereof.

The disintegrant can be selected from a group including starches such as potato starch, corn starch, wheat starch, pregelatinized starch, sodium starch glycolate; cellulose derivatives such as croscarmellose sodium or microcrystalline cellulose; polyvinylpyrrolidone; crospovidone; alginic acid and its salts; clays such as xanthan gum or Veegum; ion exchange resins or a combination thereof.

The flavoring agent can be selected from a group including natural aroma oils (peppermint oil, wintergreen oil, clove bud oil, parsley oil, eucalyptus oil, lemon oil, orange oil), menthol, menthane, anethole, methyl salicylate, eucalyptol, cinnamon, 1-methyl acetate, sage, eugenol, oxanone, alpha irisone, marjoram, lemon, orange, blackberry, propenyl guaetol acetyl, cinnamon, vanilla, thymol, linalol, cinnamaldehyde glycerol acetal, N-substituted p-menthane-3-carboxamide, 3,1-methoxy propane 1,2-diol or a combination thereof though it is preferably lemon, orange, blackberry flavor or a combination thereof.

The sweetener can be selected from a group including sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D-tryptophane, monoammonium glycyrrhizinate, neohesperidin dihydrochalcone, thaumatin, neotame, alitame, stevioside and cyclamates or a combination thereof though it is preferably sucralose, lactose, glucose, mannitol, sorbitol or a combination thereof.

The coloring agent can be selected from a group including carotenoids and chlorophyl or a combination thereof.

The surfactant can be selected from a group including sodium lauryl sulfate and magnesium lauryl sulfate or a combination thereof.

The antifoam agent can be selected from a group including simethicone emulsion and dimethyl siloxane, silicon oil or a combination thereof.

The stabilizing agent and/or agents can be selected from a group including antioxidants, chelating agents, alkalinizing agents and photoprotective agents.

The antioxidants can be selected from substances including butylated hydroxyanisole (BHA), sodium ascorbate, butylhydroxytoluene (BHT), sodium sulphite, gallates (such as propyl gallate), tocoferole, citric acid, malic acid, ascorbic acid, acetylcysteine, fumaric acid, lecithin, ascorbyl palmitate, ethylenediamine tetraacetate or a combination thereof.

The chelating agents can be selected from a group including disodium EDTA, edetic acid, citric acid, sodium citrate, potassium citrate or a combination thereof.

The alkalinizing agents can be selected from alkali metal salts such as sodium carbonate, sodium hydrogen carbonate, sodium hydroxide, sodium silicate, disodium hydrogen orthophosphate, sodium aluminate; alkaline earth metal salts such as calcium carbonate, calcium hydroxide, dibasic calcium phosphate, tribasic calcium phosphate, calcium sulphate, calcium acetate, calcium gluconate, calcium glycerophospate, magnesium carbonate, magnesium hydroxide, magnesium sulphate, magnesium acetate, magnesium silicate, magnesium aluminate; and primary, secondary and tertiary amines, cyclic amines; N,N′ -dibenzyl ethylenediamine, dietanolamine, ethylenediamine, meglumine, monosodium glutamate, polacrilin sodium, sodium alginate or a combination thereof.

The photoprotective agents can be selected from metal oxides such as titanium oxide, iron oxide and zinc oxide or a combination thereof.

According to the present invention, the effervescent formulation of the invention includes;
Calcium or calcium salt in the range of 5-60% by weight
Vitamin D in the range of 0.01-15% by weight
Genistein in the range of 0.1-25% by weight
Diluent in the range of 0.01-15% by weight
Effervescent couple in the range of 20-90% by weight
Other excipients in the range of 0.1-15% by weight

The expression “osteoporosis and related diseases” mentioned in scope of the present invention includes diseases such as osteoporosis; bone fracture including vertebral column and hip bones in postmenopausal women; bone fracture in men; idiopathic osteoporosis; osteoporosis resulting from various diseases; steroid and glucocorticoid-induced osteoporosis; osteopenia; osteomalacia; osteogenesis imperfecta; osteochondrodisplasia; sudeck atrophy; rheumatoid arthritis; Paget's disease; metastasis of malignant tumors to bones; hypercalcemia; or hyperthyroidism.

The expression “prophylaxis of the disease” refers to prevention of abovementioned diseases by administering the formulations to healthy people. This term also includes use of the formulation in people who are in the first stage of the disease. The expression “treatment of the disease” refers to use of the formulation of the present invention for treatment purposes in people diagnosed with osteoporosis or osteoporosis-related diseases.

All the components used in scope of the present invention are pharmaceutically acceptable. The term ‘pharmaceutically acceptable’ refers to the component's suitability for use in people; its having few or no side effects (toxicity, irritation, allergic response) and its providing the user an evident benefit.

### Example 1

The effervescent formulation of the present invention can be produced by direct compression, wet granulation and/or dry granulation methods applied conventionally.

| Component | Amount (% by weight) |
|---|---|
| Calcium carbonate | 17 |
| Vitamin D₃ | 0.1 |
| Genistein | 1 |
| Diluent | 13 |
| Effervescent couple | 65 |
| Other excipients | 3.9 |
| Total | 100 |

The present invention also relates to pharmaceutical formulations in effervescent form so to be used in the treatment and/or prevention of bone diseases particularly such as osteoporosis, osteomalacia and fibrous osteodystrophy which are mostly seen in postmenopausal period.

The effervescent formulations of the invention include genistein at least at 0.1%, preferably in the range of 0.1 to 5%, more preferably in the range of 0.1 to 2% in proportion to the total weight of the formulation.

The effervescent formulations of the invention include genistein in the range of 1 to 200 mg, preferably in the range of 1 to 100 mg per unit dosage form.

The inventor has developed the effervescent form of the active agent formulations generally aiming at women above the age of 40.

This dosage form is highly beneficial in terms of such aspects as ease of use, accurate dosing, high bioavailability.

The effervescent formulations of the invention include at least one pharmaceutically acceptable effervescent acid, at least one pharmaceutically acceptable effervescent base and optionally at least another pharmaceutically acceptable excipient in addition to genistein.

The effervescent formulations of the invention form a totally clear and drinkable solution within 2 minutes after they are put into water. The inventor has discovered that the water-solubility of the effervescent genistein formulations developed is considerably affected by the average particle size of genistein.

In the effervescent formulations of the invention, use of genistein with an average particle size (d₅₀) in the range of 1 to 300 μm, preferably in the range of 1 to 250 μm, more preferably in the range of 1 to 200 μm have led to a positive development in the water-solubility of the formulations. The term “average particle size (d₅₀)” mentioned here refers to the particle size where 50% of the particles by volume is present; and it was measured by dry method in Malvern Mastersizer 2000 S (Scirocco 2000) device.

A characteristic of the effervescent formulations of the invention is that the formulations include genistein as active agent with an average particle size (d₅₀) in the range of 1 to 300 μm, at least one pharmaceutically acceptable effervescent acid, at least one pharmaceutically acceptable effervescent base and at least another excipient.

A characteristic of the effervescent formulations of the invention is that the formulations include genistein as active agent with an average particle size (d₅₀) in the range of 1 to 250 μm, at least one pharmaceutically acceptable effervescent acid, at least one pharmaceutically acceptable effervescent base and at least another excipient.

A characteristic of the effervescent formulations of the invention is that the formulations include genistein as active agent with an average particle size (d₅₀) in the range of 1 to 200 μm, at least one pharmaceutically acceptable effervescent acid, at least one pharmaceutically acceptable effervescent base and at least another excipient.

The other excipients that can be used in the effervescent formulations of the invention can be selected from diluents, binders, sweeteners, lubricants, solvents, flavoring agents, pH regulating agents, coloring agents or combinations thereof.

The disintegrants that can be used in the effervescent formulations of the invention can be selected from a group including cellulose derivatives such as cross-linked carboxy methyl cellulose and/or its salts, microcrystalline cellulose, cellulose, hydroxypropyl cellulose, hydroxy ethyl cellulose, hydroxy propyl methyl cellulose, hydroxy propyl ethyl cellulose, methyl cellulose; sodium starch glycolate, alginic acid, sodium alginate, chitosan, colloidal silicone dioxide, starch, pregelatinized starch, polyvinylpyrrolidone, cross-linked polyvinylpyrrolidone or combinations thereof.

The lubricants that can be used in the effervescent formulations of the invention can be selected from a group including metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oil, leucine, polyethylene glycols (PEG), metallic lauryl sulphate (such as sodium lauryl sulphate, magnesium lauryl sulphate), sodium chloride, sodium benzoate, sodium acetate and talc.

The effervescent formulations of the invention include at least one lubricant in the range of 1 to 5%, preferably in the range of 1 to 4%, more preferably in the range of 1 to 3% by weight.

The binders that can be used in the effervescent formulations of the invention can be selected from starches such as potato starch, corn starch, wheat starch; sugars such as sucrose, glycose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethylcellulose, methylcellulose, ethylcellulose; polyvinylpyrrolidone (povidone); polyethylene glycol (PEG); waxes; calcium carbonate; calcium phosphate; alcohols such as sorbitol, xylitol, mannitol; and water; or combinations thereof.

The effervescent formulations of the invention include at least one binder in the range of 1 to 10%, preferably in the range of 1 to 8%, more preferably in the range of 1 to 5% by weight.

The diluents that can be used in the effervescent formulations of the invention can be selected from a group including alkali metal carbonates such as calcium carbonate; alkali metal phosphates such as calcium phosphate; alkali metal sulphates such as calcium sulphate; cellulose derivatives such as cellulose, microcrystalline cellulose, cellulose acetate; magnesium oxide, dextrin, fructose, dextrose, glyceryl palmitostearate, lactitol, kaolinite, lactose, maltose, mannitol, simethicone, sorbitol, starch, pregelatinized starch, talc, xylitol and/or anhydrates, hydrates and/or pharmaceutically acceptable derivatives or combinations thereof.

The effervescent formulations of the invention include at least one diluent at least at 1%, preferably in the range of 1 to 10%, more preferably in the range of 1 to 8% by weight.

The sweeteners that can be used in the effervescent formulations of the invention can be selected from a group including sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D-tryptophane, monoammonium glycyrrhizinate, neohesperidine dihydrochalcone, thaumatin, neotame, alitame, stevioside and cyclamates or a combination thereof.

The effervescent formulations of the invention include at least one diluent at least at 0.5%, preferably in the range of 0.5 to 1.5% by weight.

The flavoring agents that can be used in the effervescent formulations of the invention can be selected from a group including natural aroma oils (peppermint oil, wintergreen oil, clove bud oil, parsley oil, eucalyptus oil, lemon oil, orange oil), menthol, menthane, anethole, methyl salicylate, eucalyptol, cinnamon, 1-methyl acetate, sage, eugenol, oxanone, alpha irisone, marjoram, lemon, orange, blackberry, propenyl guaethol acetyl, cinnamon, vanilla, thymol, linalool, cinnamaldehyde glycerol acetal, N-substituted p-menthane-3-carboxamide, 3,1-methoxy propane 1,2-diol or a combination thereof.

The coloring agents that can be used in the effervescent formulations of the invention can be selected from a group including water-insoluble pigments, iron and titanium oxides, talc, beta carotene or combinations thereof.

The solvents that can be used in the effervescent formulations of the invention can be alcohol or alcohol mixtures or deionized water.

The effervescent bases that can be used in the effervescent formulations of the invention can be selected from a group including potassium carbonate, potassium bicarbonate, potassium citrate, potassium hydroxide, sodium carbonate, sodium hydrogen carbonate, sodium hydrogen citrate and combinations thereof.

The effervescent formulations of the invention include at least one effervescent base at least at 1%, preferably in the range of 1 to 70%, preferably in the range of 1 to 60%, more preferably by the range of 1 to 50% by weight.

The effervescent acids that can be used in the effervescent formulations of the invention can be selected from a group including acetic acid, citric acid, lactic acid, malic acid, phosphoric acid, propionic acid, tartaric acid or hydrates, anhydrates or combinations thereof.

The effervescent formulations of the invention include at least one effervescent acid at least at 20%, preferably in the range of 20 to 80%, more preferably in the range of 20 to 75% by weight.

The formulations of the invention can be produced by any of the methods in the prior art. These production methods can be wet granulation, dry granulation, dry blending; though the preferred production method for the formulations of the invention is wet granulation method.

The formulations of the invention can optionally be used in combination with another active agent or agents. The term “another active agent” used here refers to various vitamins and/or minerals necessary for human body.

The dosage forms for combined therapy can be taken separately, simultaneous or consecutively; as well as combining genistein with the other active agent or agents in a single dosage form.

The other active agent or agents that can be used together with genistein in combined therapy can be minerals such as calcium, potassium, magnesium, iron, sodium, zinc; or their salts such as carbonate, sulphate; vitamins such as vitamin A, vitamins B such as B1, B12, B6 and/or folic acid, vitamin C, vitamin D, vitamin E.

In combined therapy, one or two of the active agents mentioned above can be combined with genistein. In other words, the present invention includes binary or ternary combinations of genistein with the other active agents.

The other active agent or agents that can be used in the combined therapy can be produced with genistein by the same production method, while they can also be prepared by combining the active agent formulations after producing them separately.

The effervescent formulations prepared in accordance with the invention are used in the treatment and/or prevention of bone diseases particularly such as osteoporosis, osteomalacia and fibrous osteodystrophy, which are mostly seen in postmenopausal period.

What is claimed is:

1. A formulation comprising calcium, vitamin D, and genistein characterized in that said formulation is in effervescent form.

2. The effervescent formulation according to claim 1, characterized in that said formulation is formulated in powder, granule, pellet, micro tablet or tablet form.

3. The effervescent formulation according to claim 2, characterized in that said formulation is formulated in tablet form.

4. The effervescent formulation according to claim 1, characterized in that calcium comprised in said formulation is in salt form.

5. The effervescent formulation according to claim 4, characterized in that calcium comprised in said formulation is in carbonate, chloride, phosphate, citrate, lactate, glubionate, gluceptate, or gluconate salt form.

6. The effervescent formulation according to claim 5, characterized in that calcium salt comprised in said formulation is calcium carbonate.

7. The effervescent formulation according to claim 1, characterized in that said formulation comprises 5-60% calcium or calcium salt by weight.

8. (canceled)

9. The effervescent formulation according to claim 1, characterized in that the particle size of calcium or calcium salt is smaller than 100 μm.

10. The effervescent formulation according to claim 9, characterized in that the particle size of calcium or calcium salt is smaller than 60 μm.

11. The effervescent formulation according to claim 1, characterized in that vitamin D is vitamin D₂ (ergocalciferol), vitamin D₃ (cholecalciferol), or a combination thereof.

12. The effervescent formulation according to claim 11, characterized in that vitamin D is vitamin D₃ (cholecalciferol).

13. The effervescent formulation according to claim 1, characterized in that said formulation comprises 0.01-15% vitamin D by weight.

14. The effervescent formulation according to claim 1, characterized in that the amount of vitamin D is in the range of 200-1000 IU.

15. The effervescent formulation according to claim 1, characterized in that said formulation comprises 0.1-25% genistein by weight.

16. (canceled)

17. The effervescent formulation according to claim 1, characterized in that said formulation comprises pharmaceutically acceptable excipients in addition to calcium, vitamin D, and genistein.

18. The effervescent formulation according to claim 17, characterized in that said formulation comprises one or more excipients, wherein said excipient is an effervescent couple, diluent, binder, glidant, lubricant, disintegrant, flavoring agent, sweetener, coloring agent, surfactant, anti-foam agent, or stabilizing agent.

19. The effervescent formulation according to claim 18, characterized in that the effervescent couple is composed of an effervescent acid and an effervescent base.

20. The effervescent formulation according to claim 19, wherein the effervescent acid is selected from the group consisting of acetic acid, citric acid, lactic acid, malic acid, phosphoric acid, propionic acid, and tartaric acid, and combinations thereof.

21. The effervescent formulation according to claim 20, characterized in that the effervescent acid is citric acid, malic acid, or a combination thereof.

22. The effervescent formulation according to claim 19, wherein the effervescent base is selected from the group consisting of potassium carbonate, potassium bicarbonate, potassium citrate, potassium hydroxide, sodium carbonate, sodium hydrogen carbonate, and sodium hydrogen sulfate, and combinations thereof.

23. The effervescent formulation according to claim 22, characterized in that the effervescent base is sodium carbonate, sodium hydrogen carbonate, or a combination thereof.

24-37. (canceled)

38. The effervescent formulation according to claim 1 characterized in that said formulation comprises calcium or calcium salt in the range of 5-60% by weight, Vitamin D in the range of 0.01-15% by weight, genistein in the range of 0.1-25% by weight, diluent in the range of 0.01-15% by weight, effervescent couple in the range of 20-90% by weight, and other excipients in the range of 0.1-15% by weight.

39. (canceled)

40. An effervescent formulation, characterized in that said formulation comprises genistein with an average particle size (d₅₀) in the range of 1 to 300 μm as active agent, at least one pharmaceutically acceptable effervescent acid, at least one pharmaceutically acceptable effervescent base, and at least another excipient.

41. The effervescent formulation according to claim 40, characterized in that said formulation comprises genistein with an average particle size (d₅₀) in the range of 1 to 250 μm as active agent, at least one pharmaceutically acceptable effervescent acid, at least one pharmaceutically acceptable effervescent base, and at least another excipient.

42. The effervescent formulation according to claim 40, characterized in that said formulation comprises genistein with an average particle size (d₅₀) in the range of 1 to 200 μm as active agent, at least one pharmaceutically acceptable effervescent acid, at least one pharmaceutically acceptable effervescent base, and at least another excipient.

43-49. (canceled)

50. The effervescent formulation according to claim 40, characterized in that said formulations comprise at least one effervescent acid in the range of 20 to 80% by weight.

51. The effervescent formulation according to claim 40, characterized in that said formulations comprise at least one effervescent acid in the range of 20 to 75% by weight.

52. The effervescent formulation according to claim 40, characterized in that said formulation comprises genistein at least at 0.1% by weight.

53. The effervescent formulation according to claim 52, characterized in that said formulation comprises genistein in the range of 0.1 to 5% by weight.

54. The effervescent formulation according to claim 52, characterized in that said formulation comprises genistein in the range of 0.1 to 2% by weight.

55. The effervescent formulation according to claim 40, characterized in that said formulation comprises a second active agent.

56. The effervescent formulation according to claim 55, wherein the second active agent is selected from the group consisting of minerals such as calcium, potassium, magnesium, iron, sodium, zinc; or their salts such as carbonate, sulphate; vitamins such as vitamin A, vitamins B such as B1, B12, B6 and/or folic acid, vitamin C, vitamin D, and vitamin E, and combinations thereof.

57. A production method for the production of an effervescent formulation comprising genistein with an average particle size (d₅₀) in the range of 1 to 300 μm, at least one pharmaceutically acceptable effervescent acid, at least one pharmaceutically acceptable effervescent base and at least another excipient, characterized in that said method is a wet granulation method.

58. A method of treating or preventing bone disease in a subject, said method comprising administering to said subject a therapeutically effective amount of the effervescent formulation according to claim 1.

59. The method according to claim 58, wherein the bone disease is osteoporosis, osteomalacia, or fibrous osteodystrophy.

60. The method according to claim 58, wherein the subject is in a postmenopausal period.

61. A method of treating or preventing bone disease in a subject, said method comprising administering to said subject a therapeutically effective amount of the effervescent formulation according to claim 40.

62. The method according to claim 61, wherein the bone disease is osteoporosis, osteomalacia, or fibrous osteodystrophy.

63. The method according to claim 61, wherein the subject is in a postmenopausal period.
